(19) United States
(12) Patent Application Publication
(10) Pub. No.: US 2019/0110915 A1
(43) Pub. Date: Apr. 18, 2019

(54) INTRODUCTION ELEMENT FOR A MEDICAL INTRODUCTION DEVICE

(71) Applicant: BIOTRONIK AG, Buelach (CH)

(72) Inventor: Mark Keller, Aarau (CH)

(21) Appl. No.: 16/157,571

(22) Filed: Oct. 11, 2018

(30) Foreign Application Priority Data
Oct. 13, 2017 (EP) 17196410.9

(51) Int. Cl. A61F 2/962

(52) U.S. Cl. CPC A61F 2/962

(57) ABSTRACT

An implant introduction device designed to position a medical implant at a destination in a human or animal body. A sleeve extends in an axial direction has at least a first section and a second section opposite the first section in the axial direction. The two sections are connected with one another through a connection area around the sleeve in the peripheral direction. The first section is made of a first material and the second section is made of a second material, the first material having higher bending rigidity than the second material. The connection area defines a meandering course in the peripheral direction.

### PRIORITY CLAIM

This application claims priority under 35 U.S.C. § 119 and all applicable statutes and treaties from prior European Application EP 17196410.9, filed Oct. 13, 2017.

### FIELD OF THE INVENTION

A field of the invention is implant introduction devices for implanting medical devices into a body via catheter.

### BACKGROUND

In medicine, it is common to use implants that are put into an animal and/or human body to perform replacement functions permanently, or at least for a longer period of time. These implants are, e.g., heart implants, vascular prostheses, stents, or other suitable implants, which are connected with an introduction device, in particular a catheter, before introduction into the body and are, with its help, accurately placed at the operating site and released in a defined manner.

The implants are transferred into the animal and/or human body using, e.g., a tubular introduction element, through which the implant is pushed by means of the introduction device.

During the so-called tracking, that is, as the introduction element is being pushed into a body lumen, the introduction element or the introduction device travels on a path that has various anatomically determined curvatures in space. In order to be able to travel on this curved path with as little friction as possible, the systems have, among other things, variable bending rigidity along their length, i.e., in the axial direction. As a rule, this is accomplished by selecting different plastics with various flexural moduli (moduli of elasticity).

However, this presents a problem that it causes a discontinuity in rigidity in a transitional zone (material transition or joint) in the introduction element or the catheter tube, which represents a weak point for the buckling and kinking damage mechanism.

The rigidity of an introduction element or catheter can be varied not only by using plastics having different bending rigidities, but also by putting braided tubes or braids into the catheter wall as armor, whose pitch (braid angle) can once again be varied. When this is done, the material and dimension of the braid wire remain constant, or can be gradually varied only using technically very elaborate methods/processes.

### SUMMARY

A preferred embodiment provides an implant introduction device with a sleeve that extends in an axial direction having at least a first section and a second section opposite the first section in the axial direction. The two sections are connected with one another through a connection area around the sleeve in the peripheral direction. The first section is made of a first material and the second section is made of a second material. The first material has higher bending rigidity than the second material. The connection area has a meandering course in the peripheral direction.

### BRIEF DESCRIPTION OF THE DRAWINGS

Features and advantages of the invention are explained on the basis of the description of the figures, which show sample embodiments of the invention. The figures are as follows:

FIG. 1 a schematic view of an inventive introduction element;

FIG. 2A-2C schematic representations of possible other meandering courses of the connection area; and

FIG. 3 a view of an introduction device in which the inventive introduction element can be used.

### DETAILED DESCRIPTION OF THE PREFERRED EMBODIMENTS

An implant introduction device of a preferred embodiment is designed to position a medical implant at a destination in a human or animal body. The introduction element has a sleeve that extends in an axial direction and that surrounds an axially extending lumen or interior of the sleeve, the sleeve having at least one preferably tubular first section and a (preferably tubular) second section opposite the first section in the axial direction, the two sections being connected with one another through a connection area around the sleeve in the peripheral direction and in which the two sections change into one another, the first section being made of a first material and the second section being made of a second material, the first material having higher bending rigidity than the second material. The invention provides that the connection area has a meandering course in the peripheral direction.

Here it is preferably provided that the meandering course of the connection area is formed by mountain-shaped and valley-shaped sections of the connection area arranged one after the other in alternation in the peripheral direction of the sleeve. It is also preferably provided that the two sections in the connection area be welded together.

In a preferred embodiment, the implant introduction devices is built from two areas that are connected together through a connection area. In other embodiments, it is also possible for more than two areas to be connected together so that two outer areas are provided and are connected together through at least one inner area. Thus, in the case of three areas, there are two outer areas that are connected through an inner area as described here. Accordingly, in the case of four areas, there are two outer areas that are connected through two inner areas as described here. It is preferred that all areas have a different flexural modulus, preferably converging in one direction, that is, graduated from not very flexible to flexible. However, there are also other conceivable embodiments, as will be described in detail in another place here.

The invention has the advantageous effect that the transitional area or the connection area between the at least two sections or materials has a not insignificant component in the axial direction of the sleeve. Thus, the result is that the hardness or bending rigidity of the two sections undergoes a gradual transition in the axial direction, this transition extending along the length (in the axial direction) from the lowest valley to the highest mountain of the meandering connection area, i.e., the length of the gradual transition can advantageously be adjusted through the extension of the meandering course in the axial direction. Furthermore, the meandering course also advantageously makes it possible to achieve a certain cylindrical symmetry (in the axial direction) of the bending rigidity transition, e.g., by providing a sufficient number of mountain-shaped and valley-shaped sections of the meandering course, that is, of the connection area.

A preferred embodiment provides that a base of the respective mountain-shaped section be smaller than its height. The base of these mountain-shaped sections lies at half height between the highest point of a mountain-shaped section and the lowest point of an adjacent valley-shaped section.

A preferred embodiment of the invention further provides that every mountain-shaped section is formed by two legs each, each pair of legs running toward one another starting from the base and including an angle that preferably lies in the range from 1° to 60°, more preferably in the range from 2 to 45°, and most preferably in the range from 5 to 30°. Furthermore, each of the mountain-shaped sections can also be formed by two legs each, each pair of legs of the respective valley-shaped section including an angle that preferably lies in the range from 1° to 60°, more preferably in the range from 2 to 45°, and most preferably in the range from 5 to 30°.

Each of the individual valley-shaped and mountain-shaped sections of the connection area can be rectangular, trapezoidal, triangular, or have a continuous curvature, a continuous curvature, especially a wave-shaped course of the connection area being preferred.

One embodiment of the inventive implant introduction device provides that the first material be a plastic or have a plastic, the said plastic especially being one of the following plastics: a thermoplastic elastomer, a polyether block amide (PEBA), a thermoplastic polyurethane, a polycarbonate-based thermoplastic polyurethane, or a polyether-based thermoplastic polyurethane. PEBA is a thermoplastic elastomer, namely a block copolymer based on a polyamide (e.g., PA6, PA11, PA12).

One embodiment of the inventive implant introduction device further provides that the second material be a plastic or have a plastic, the said plastic especially being one of the following plastics: a thermoplastic elastomer, a polyether block amide (PEBA), a thermoplastic polyurethane, a polycarbonate-based thermoplastic polyurethane, or a polyether-based thermoplastic polyurethane.

One embodiment of the inventive implant introduction device further provides that the first material have a flexural modulus that lies in the range from 7 MPa to 650 MPa.

One embodiment of the inventive implant introduction device further provides that the second material have a flexural modulus in the range from 7 MPa to 650 MPa, this flexural modulus being different from that of the first material.

In one embodiment there is a difference of at least 5 MPa between the flexural modulus of the first material and that of the second material. In another embodiment, the difference between the flexural modulus of the first material and that of the second material is in the range from 5 to 350 MPa, more preferably in the range from 20 to 250 MPa, and most preferably in the range from 25 MPa to 180 MPa.

One embodiment of the inventive implant introduction device further provides that the first section and the second section be connected together over or in the connection area by material bonding, especially by fusing of the two materials. Accordingly, it is preferred that the materials of the first section and of the second section be selected so that the materials can be welded together. Welding the first and the second sections together has the first advantage that it introduces a strong bonding connection between the areas, and the second advantage that this connection can advantageously be produced by an automated process, and therefore it is economical.

The connection area can also be referred to as a transitional area, since here there is a change from the first to the second material (e.g., caused by the fusion).

Another aspect of this invention involves providing an introduction device that has an inventive implant introduction device, i.e., one described here, and that is designed to position and especially to release a medical implant at a destination in a human or animal body, the implant introduction device forming at least one of the following components of the introduction device or forming at least a part of one of the following components of the introduction device:
An inner shaft to carry the implant, the inner shaft being guided especially in an outer shaft of the introduction device;
An outer shaft that surrounds an inner shaft of the introduction device, this inner shaft being designed to carry the implant;
a lock through which it is possible to introduce, into a body lumen of a human or animal body, an outer shaft of the introduction device together with an inner shaft of the introduction device, this inner shaft being guided in the outer shaft and serving to carry the implant;
a stabilizer that serves to stabilize an outer shaft of the introduction device and that preferably surrounds this outer shaft, an inner shaft of the introduction device being guided in this outer shaft to carry the implant.

It is preferably provided that the respective less hard second section form a respective distal section of the respective component, i.e., a section that is arranged further away, along the introduction device, from an operator or a handling device of the introduction device, than is a proximal section of the component. The introduction device is especially configured to allow the operator to operate the introduction device by means of the handling device, which has, in particular, suitable means of actuation for this purpose.

Another aspect of this invention discloses a production process for an inventive implant introduction device, this process comprising the following steps:
Providing a first and a second section of a sleeve of an implant introduction device to be produced, the sections preferably being tubular, the first section being made of a first material and the second section being made of a second material, the first material having a higher bending rigidity than the second material, the first section having a face that goes around in a meandering course, and the second section having a face with a meandering course that is complementary to that of the first face, so that the two faces can lie against one another in a form-fit manner;
Arranging the two sections together in such a way that the two faces lie against one another in a form-fit manner; and
Forming a connection between the two faces, preferably by material bonding, so that the two sections are connected together into a single-piece sleeve of the implant introduction device, this sleeve surrounding, in particular, a continuous lumen that extends from the first section into the second section of the sleeve.

One embodiment of the inventive process provides that the said (especially material bonding) connection be produced by fusing together the two faces.

That is, the (especially material bonding) connection creates the said meandering connection or transitional area of the sleeve, this connection or transitional area having, in particular, the above-mentioned features or properties.

Preferably the meandering course of the faces, which imparts to the respective face or the respective section, in particular, a crown-like geometry, is cut by means of a laser into the ends of the respective section, which have not yet been put in their final form.

The sections are then preferably put onto a first mandrel, the faces being prefixed against one another by means of addition of heat. The first mandrel can be variable in diameter (e.g., it can be conical). The mandrel can be provided by any suitable bodies (especially those variable in diameter), e.g., even balloons, etc.

After that, the sleeve formed from the two sections is preferably put onto a second mandrel that has or predetermines the final diameter of the sleeve, the sleeve preferably being thermally applied onto one or more layers lying under the sleeve by means of heat-shrink tubing, e.g., onto a PTFE liner, a laser-structured metal or plastic pipe, and/or a braid. The second mandrel can also be variable in diameter (e.g., conical). In particular, the heat-shrink tubing is taken back off after that.

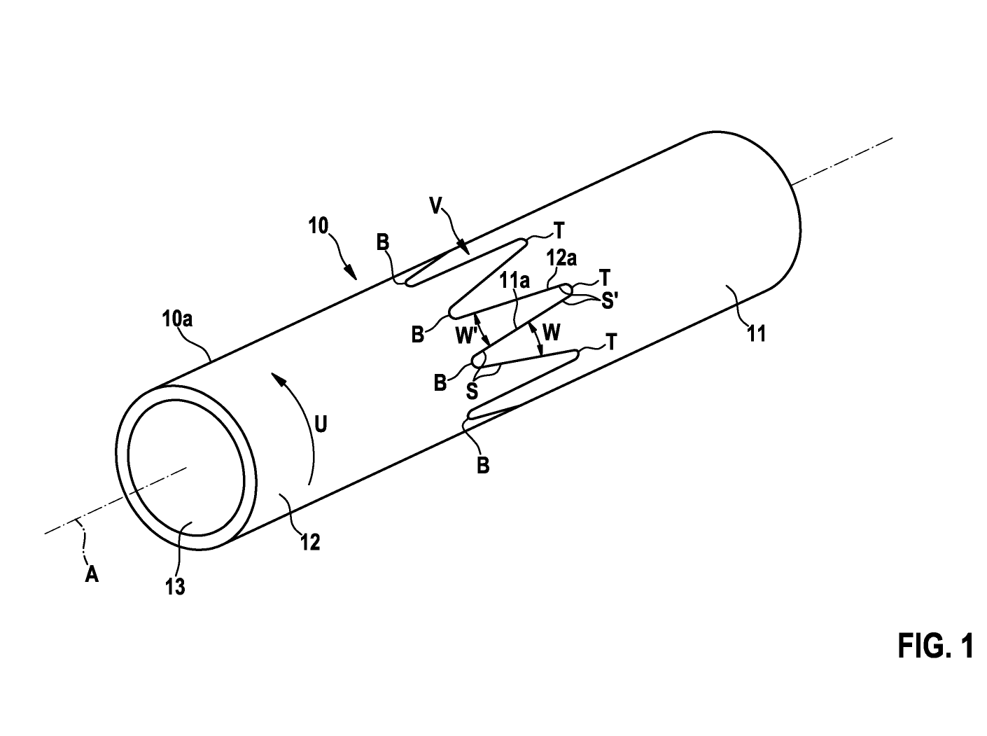

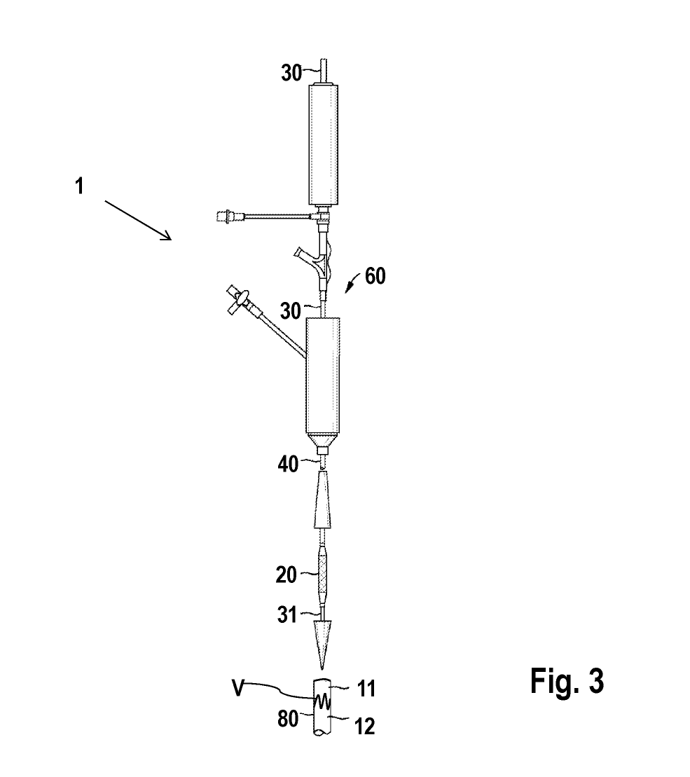

FIG. 1 shows an embodiment of an inventive implant introduction device 10 that can be used in an introduction device 1, e.g., according to FIG. 3, this introduction device 1 being designed to position a medical implant 20 at a destination in a human or animal body.

As is shown in FIG. 1, the implant introduction device 10 has a, e.g., a hollow cylindrical or hollow conical sleeve 10a that extends in an axial direction A, that surrounds a continuous lumen 13, and that has a first section 11 and a second section 12 that is opposite the first section 11 in the axial direction A. Here the two sections 11, 12 are connected together through a connection area V going around the sleeve 10a in its peripheral direction U, the first section 11 changing into the second section 12 in the connection area (in the axial direction A). Furthermore, the first tubular section 11 is made of a first material and the second tubular section 12 is made of a second material, the first material having a higher flexural modulus than the second material. The invention now provides that the said connection area V goes around in the peripheral direction U in a meandering course. Here the connection area or transitional area V with a meandering course has mountain-shaped and valley-shaped sections B, T that are arranged in alternation in the peripheral direction U.

According to FIG. 1, each of these sections of the connection area V can have a continuous curvature, resulting, in particular, in a connection area V that has a wave-shaped course, as is shown in FIG. 1.

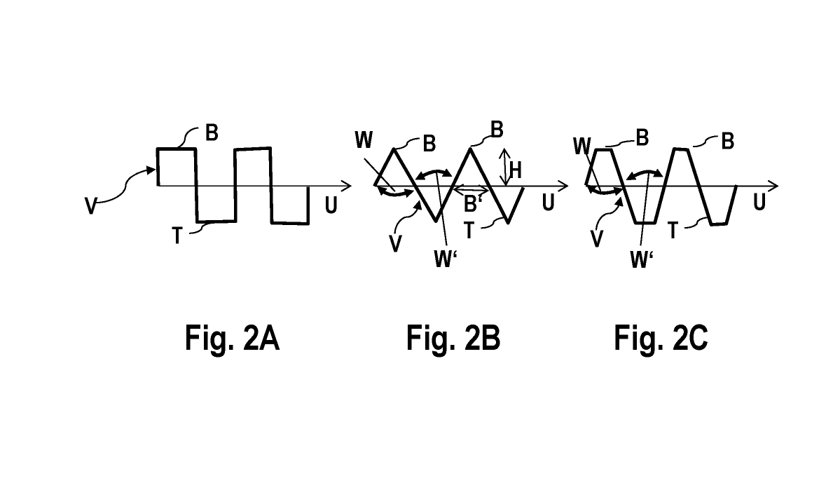

However, other meandering courses or geometries are also conceivable, as are indicated, e.g., in FIGS. 2A, 2B, and 2C. As shown in these figures, the connection area V can also have a meandering course with rectangular mountain-shaped or valley-shaped sections B, T (FIG. 2A), triangular mountain-shaped or valley-shaped sections B, T (FIG. 2B), or trapezoidal mountain-shaped or valley-shaped sections B, T (FIG. 2C). Other modifications of these theoretically possible courses are also conceivable.

It is preferably provided, as is indicated in FIGS. 1, 2B, and 2C, that every mountain-shaped section B is formed by two legs S each, each pair of legs S of the respective mountain-shaped section B being able to form or include an angle W that preferably lies in the range from 1° to 60°, more preferably in the range from 2 to 45°, and most preferably in the range from 5 to 30°. In the same way, each mountain-shaped section B can be formed by two legs S′ each, each pair of legs S′ of the respective valley-shaped section T forming an angle W′ that preferably also lies in the range from 1° to 60°, more preferably in the range from 2 to 45°, and most preferably in the range from 5 to 30°.

Furthermore, in principle it can be provided, as indicated in the example in FIG. 2B, that the respective base B′ of a mountain-shaped section B be smaller than its height H. The base B′ corresponds to the foot of the respective mountain-shaped section B and has a width that corresponds to the separation of the legs S of the respective mountain-shaped section B at half height between the highest point of the mountain-shaped section B and the lowest point of the adjacent valley-shaped sections T. The height H is the distance from the base B to the highest point of the mountain-shaped section B. That is, the height runs perpendicular to the peripheral direction U and parallel to the axial direction A.

It is preferably further provided that the first material be a plastic or have a plastic, the said plastic especially being one of the following plastics: a thermoplastic elastomer, a polyether block amide, a thermoplastic polyurethane, a polycarbonate-based thermoplastic polyurethane, or a polyether-based thermoplastic polyurethane. Here the first material can have a flexular modulus that lies in the range from 7 MPa to 650 MPa.

It is preferably further provided that the second material be a plastic or have a plastic, the said plastic especially being one of the following plastics: a thermoplastic elastomer, a polyether block amide, a thermoplastic polyurethane, a polycarbonate-based thermoplastic polyurethane, or a polyether-based thermoplastic polyurethane. Here it can be further provided that the second material have a flexular modulus in the range from 7 MPa to 650 MPa, this flexural modulus being different from that of the first material.

In one usage form the area of the first material lies proximal to the operating surgeon, that is away from the patient, and the first material has a higher flexural modulus than the second material. In another usage form the area of the first material lies distal to the operating surgeon, that is away from the patient, and the first material has a higher flexural modulus than the second material. The preferred variant is the one in which the less flexible area is the one that is used proximally. Furthermore, other embodiments are also conceivable that have more than two areas, for example three areas and that provide sequences such as hard-soft-hard or soft-hard-soft, where hard means “less flexible” and soft means “more flexible”.

Furthermore, it is preferable for the connection of the first section 11 with the second section 12 in the connection area V to be made by material bonding, e.g., by fusing the two sections or materials. Other types of connection are also conceivable, in particular connections by substance bonding.

To accomplish this, the two sections 11, 12 can first be prepared in separate form, the respective section 11, 12 of the sleeve 10a to be produced being given a correspondingly meandering face 11a or 12a, each of which follows the course of the later connection area V. The two faces 11a, 12a have a complementary shape, so that they preferably can be arranged against one another in a form-fit manner, the two sections 11, 12 aligning with one another or lying opposite one another in the direction of the longitudinal axis or in the axial direction A.

In this arrangement of the two sections 11, 12 to one another (see FIG. 1), it is now possible to produce a material bonding connection or another connection between the two faces 11a, 12a, so that the two sections 11, 12 are connected or fused together into a single-piece sleeve 10a of the implant introduction device 10, forming the said connection area V (see above).

Finally, for further illustration of the invention, FIG. 3 shows an introduction device 1 for positioning and especially for releasing a medical implant 20 at a destination in a human or animal body, this introduction device 1 allowing the use of the inventive implant introduction device 10.

The introduction device 1 comprises an inner shaft 30 and, surrounding this inner shaft 30, an outer shaft 40 at whose proximal ends there is a usual handling device 60 (not described in detail) with various connection pieces, e.g., for irrigating the lumen of or the lumen between inner shaft 30 and outer shaft 40, and for releasing the implant 20. Between the opposite distal ends, there is an implant 20 with a usual guide device 31 on the inner shaft 30.

To introduce the catheter or the ensemble of outer shaft 40, the inner shaft 30 surrounded by the outer shaft 40, the implant 20 arranged on the inner shaft 30 with guide device 31, into the human or animal body, this catheter with the implant 20 or the guide device 31 is pushed forward, e.g., through a lock 80, which has previously be introduced into the blood vessel in question, so that the catheter can slide through the lock 80 into the blood vessel.

With the introduction device 1 shown in FIG. 3, it is now possible in principle to use the inventive implant introduction device 10 for all tubular components.

For instance, the inventive implant introduction device 10 can be used, e.g., as inner shaft 30 or outer shaft 40, or at least form a section or a part of such a shaft.

Furthermore, it is also conceivable to use the implant introduction device 10 as lock 80 or as a section or part of the lock 80, as is indicated in FIG. 3.

Furthermore, in an introduction device 1 it is also possible to use the implant introduction device 10 as a stabilizer for the outer shaft 40, this stabilizer surrounding the outer shaft 50 (not shown in FIG. 2).

The inventive solution provides a simple, economical, and secure process for changing the bending rigidity of an implant introduction device 10 gradually in the axial direction A, advantageously making it possible to avoid the discontinuities in rigidity that usually occur.

It will be apparent to those skilled in the art that numerous modifications and variations of the described examples and embodiments are possible in light of the above teaching. The disclosed examples and embodiments may include some or all of the features disclosed herein. Therefore, it is the intent to cover all such modifications and alternate embodiments as may come within the true scope of this invention.

What is claimed is:

1. An implant introduction device, comprising: a sleeve that extends in an axial direction having at least a first section and a second section opposite the first section in the axial direction, the two sections being connected with one another through a connection area around the sleeve in the peripheral direction, the first section being made of a first material and the second section being made of a second material, the first material having higher bending rigidity than the second material, wherein the connection area has a meandering course in the peripheral direction.

2. An introduction element according to claim 1, wherein the meandering course of the connection area is formed by mountain-shaped and valley-shaped sections of the connection area arranged in alternation in the peripheral direction.

3. An implant introduction device according to claim 2, wherein a base of the respective mountain-shaped section is smaller than its height.

4. An implant introduction device according to claim 2, wherein every mountain-shaped section is formed by two legs each, each pair of legs of the respective mountain-shaped section forming an angle in the range from 5° to 60°,

5. An implant introduction device according to claim 2, wherein every mountain-shaped section is formed by two legs each, each pair of legs of the respective valley-shaped section forming an angle in the range from 1° to 60°.

6. An implant introduction device according to claim 2 wherein each of the valley-shaped and mountain-shaped sections is rectangular, trapezoidal, triangular, or has a continuous curvature.

7. An implant introduction device according to claim 1, wherein the first material comprises a plastic selected from the group of a polyether block amide, a thermoplastic polyurethane, a thermoplastic elastomer, a polycarbonate-based thermoplastic polyurethane, and a polyether-based thermoplastic polyurethane.

8. An implant introduction device according to claim 7, wherein the second material comprises a plastic selected from the group of a polyether block amide, a thermoplastic polyurethane, a thermoplastic elastomer, a polycarbonate-based thermoplastic polyurethane, and a polyether-based thermoplastic polyurethane.

9. An implant introduction device according to claim 1, wherein the first material has a flexular modulus that lies in the range from 7 MPa to 650 MPa.

10. An implant introduction device wherein the first section and the second section are connected together over the connection area by material bonding.

11. The implant introduction device according to claim 10, wherein the material bonding comprises fusing of the two materials.

12. The implant introduction device according to claim 1, wherein the implant introduction device comprises an inner shaft to carry an implant.

13. The implant introduction device according to claim 1, wherein the implant introduction device comprises an outer shaft that surrounds an inner shaft that carries an implant.

14. The implant introduction device according to claim 1, wherein the implant introduction device comprises a lock configured to introduce an outer shaft together with an inner shaft that carries an implant into a body lumen.

15. The implant introduction device according to claim 1, wherein the implant introduction device comprises a stabilizer that serves to stabilize an outer shaft of the that surrounds an inner shaft that carries an implant.

16. A production process for an implant introduction device comprising the following steps:
providing at least a first and a second section of a sleeve, the first section being made of a first material and the second section being made of a second material, the first material having a higher bending rigidity than the second material, the first section having a face that defines a meandering course, and the second section having a face that defines a meandering course that is complementary to that of the first face so that the two faces can lie against one another in a form-fit manner;
arranging the two sections together in such a way that the two faces lie against one another in a form-fit manner; and
forming a connection between the two faces by material bonding so that the two sections are connected together into a single-piece sleeve.

17. A process according to claim 16, wherein the material bonding comprises fusing the two faces.
